(19) United States
(12) Patent Application Publication
(10) Pub. No.: US 2004/0071674 A1
(43) Pub. Date: Apr. 15, 2004

(54) Therapeutic treatment of digestive organs with engineered stem cells

(76) Inventors: Hiroshi Mashimo, Lexington, MA (US); Satish K. Singh, Sharon, MA (US)

Correspondence Address: WEINGARTEN, SCHURGIN, GAGNEBIN & LEBOVICI LLP, TEN POST OFFICE SQUARE, BOSTON, MA, 02109, US

(21) Appl. No.: 10/392,377

(22) Filed: Mar. 19, 2003

Related U.S. Application Data
(60) Provisional application No. 60/365,155, filed on Mar. 19, 2002.

(51) Int. Cl. A61K 48/00; C12N 5/06; C12N 5/08

(52) U.S. Cl. 424/93.21; 435/354; 435/366

(57) ABSTRACT

A method of directly implanting embryonic or adult somatic stem cells into a desired site in a mammal, e.g., into the adult digestive tract, for gene therapy and/or cell replacement is disclosed. Stem cells with their inherent pluripotency have been found to be capable of being implanted directly, e.g., in the digestive system where they become permanently engrafted. The advantage of cultured stem cells is that they can be expanded in vitro and engineered to produce cells that stably express a variety of gene products. Stem cells directly implanted in the gastrointestinal tract according to the method of the invention, have been shown to differentiate into specialized cells of the adult gut (such as nerves, muscles and epithelia). These cells remain at the site of injection and respect local tissue architecture while permanently expressing transfected genes, e.g., nitric oxide synthase, which catalyzes the production of nitric gas in vivo. This approach is particularly well suited for such a diffusible, unstable and transiently bioactive compound as nitric oxide. This technology is useful, e.g., in treating gastrointestinal disorders where key components of a normally functioning bowel are missing, and where local administration of the gene product is thought to be essential.

### CROSS REFERENCE TO RELATED APPLICATIONS

This application claims the priority of U.S. Provisional Application No. 60/365,155 filed Mar. 19, 2002 entitled, ENGINEERED STEM CELLS DELIVERED DIRECTLY INTO DIGESTIVE ORGANS FOR DRUG DELIVERY, GENE THERAPY, CELL REPLACEMENT, RESEARCH AND DIAGNOSTIC APPLICATIONS, the whole of which is hereby incorporated by reference herein.

### STATEMENT REGARDING FEDERALLY SPONSORED RESEARCH OR DEVELOPMENT

Part of the work leading to this invention was carried out with United States Government support from the Department of Veterans Affairs through National Institutes of Health Grant Nos. K08 DK02410-05 and R03 DK56079-03. Therefore, the U.S. Government has certain rights in this invention.

### BACKGROUND OF THE INVENTION

The traditional approaches to gene therapy (e.g., using adenovirus, lentivirus, and HSV) have met with mixed success. Major disadvantages of these approaches to gene therapy are the creation of inflammation at the site of delivery, the variable uptake and expression in different tissues, and the decline in expression of the gene of interest over time. The gastrointestinal tract has proven particularly problematic with respect to these approaches because it is immunologically highly active and because the entire epithelium is shed and renewed within days. It has been found that expression of a reporter gene introduced to the gut by a replication-defective adenovirus occurred largely in areas of Peyer's patches, perhaps reflecting the access through M cells (Foreman, 1998). Gene transfer by lipofection and adenoviral vectors can be carried out in gut epithelial cells such as for treatment of hemophilia (Lozier, 1997). However, local and stable long-term in vivo expression of genes in the gastrointestinal tract has not been achieved by any method.

One particularly intractable set of digestive diseases is caused by nitric oxide deficiency. A relative deficiency of nitric oxide (NO), a readily diffusible gas, is implicated in a variety of gastrointestinal motility disorders including achalasia, Chagas' disease, diabetic gastroparesis, Hirschsprung's disease, sphincter of Oddi dysfunction and infantile hypertrophic pyloric stenosis. Amyl nitrate, nitroglycerin and, more recently, sildenafil, all of which are believed to work by augmenting the NO pathway, have been used to diagnose or treat some of these conditions in the esophagus, as well as in other sphincteric regions of the gastrointestinal tract. Unfortunately, these pharmacological agents are transient in effect and have wide systemic side effects. Thus, if it were possible to restore the local expression of nitric oxide in a stable, long term manner, significant progress could be made in treating these dysfunctions.

### BRIEF SUMMARY OF THE INVENTION

Described herein are methods for preparing and directly implanting engineered embryonic (ES) or somatic (SS) stem cells into a desired site in a mammal, e.g., into the adult digestive tract, to accomplish the above objectives by gene therapy and/or cell replacement. Stem cells with their inherent pluripotency (ability to differentiate into a variety of tissues) are capable of being implanted directly, e.g., in the digestive system where they become permanently engrafted. The advantage of cultured ES and SS cells is that they can be expanded in vitro and engineered to produce cells that stably express a variety of gene products.

We have demonstrated that embryonic and adult somatic, e.g., bone marrow and intestine-derived, stem cells directly implanted in the gastrointestinal tract according to the method of the invention, can differentiate into specialized cells of the adult gut (such as nerves, muscles and epithelia). These cells remain at the site of injection and respect local tissue architecture while expressing transfected genes permanently. These data demonstrate that undifferentiated embryonic and adult somatic stem cells transfected with specific genes, e.g., a nitric oxide synthase, which catalyzes the production of nitric gas in vivo, survive in vivo in the adult intestine and show great promise for a novel method of drug delivery. This approach is particularly well suited for such a diffusible, unstable and transiently bioactive compound as nitric oxide. This technology is useful, e.g., in treating gastrointestinal disorders where key components of a normally functioning bowel are missing, and where local administration of the gene product is thought to be essential. Examples of such disorders include inflammatory bowel disease, Crohn's disease, ulcerative colitis, gastroparesis, infantile pyloric stenosis, Hirschsprung's disease, and diabetes.

### BRIEF DESCRIPTION OF THE SEVERAL VIEWS OF THE DRAWINGS

Other features and advantages of the invention will be apparent from the following description of the preferred embodiments thereof and from the claims, taken in conjunction with the accompanying drawings, in which:

FIG. 1 shows X-gal staining of the pylorus 60 days post-injection according to the method of the invention;

FIGS. 2A and 2B are cross sections of the pylorus in the stained region shown in FIG. 1. FIG. 2A shows specific staining in the mucosal and smooth muscle layers while staining on the mucosa alone is shown in FIG. 2B;

FIGS. 3A and 3B show a comparison of X-gal staining (3A) and H&E staining (3B) in the same general region of the pylorus;

FIGS. 4A-4C show, in high power detail, staining of the antrum in cross section four weeks after injection with ES cells bearing iNOS: (A) antrum stained with epithelial specific anti-e-cadherin antibody; (B) a red pseudocolor DIC image of X-gal staining; (C) antibody and X-gal staining co-localized within gastric glands (yellow); and

FIGS. 5A-5B show that four weeks after injection of the duodenum (with ES cells bearing iNOS), blue ROSA26-derived stem cells are present (5A), largely within glands that are cytokeratin positive (5B).

### DETAILED DESCRIPTION OF THE INVENTION

Stem cell technology enables us to manipulate the genome and to “load” the stem cells with particular genes for constitutive expression and local delivery of their product. For example, as described herein, a gene encoding nitric oxide synthase was transfected into mouse embryo stem cells. Its product, NO, has a very short biological half-life and is a readily diffusible gas. Conventional pharmacological methods for chronic local delivery of such a substance would be impossible. Several key conditions were required in order for the method of the invention to be carried out successfully. The stem cells needed to be syngeneic with the host animal so as not to be rejected immunologically. They needed to harbor an endogenous marker, such as β-galactosidase, so that injected cells could be distinguished from the host animal tissues. Finally, they had to be manipulable in vitro for stable expression the gene of interest and to express a functional gene product, i.e., production of NO gas.

We have demonstrated that, once injected in adult tissues, the stem cells survived in the host, incorporating into the local tissue architecture. Totipotent cells did not form tumors, as observed by others when choriocarcinoma cell lines were injected into the peritoneal cavity. In addition, the stem cells remained at or near the injected site and did not migrate as was noted previously when stem cells were injected in embryonic gut (Natarajan, 1999).

The source of human embryonic stem cells has become a highly publicized and controversial ethical issue. The future use of ES in humans would be severely restricted by considerations of histoincompatibility and probable need for immune suppression, as with most tissue transplants. Thus, a more appealing approach is to use a patient's own syngeneic tissues for cell replacement and possible gene therapy. Fortunately, many tissues have a subpopulation of pluripotent “stem cells” that give rise to a range of cells and may regulate the expansion and turnover of the cellular compartments typical of the tissue in which they reside. Such stem cell populations have been identified in the brain (Vescovi, 1999), bone marrow (Bianco, 2001), liver (Michalapoulos, 2000), intestine (Booth, 2000), umbilical cord blood (Moore, 2000), and fat (Zuk, 2001). Indeed, it is possible that virtually every tissue has a small population of pluripotent stem cells that are able to regulate the cell population and enable regeneration. More recently, in vitro cultures and in vivo transplantation assays have indicated that adult somatic stem cells can give rise to developmentally unrelated tissues. For example, brain stem cells can adopt multiple fates and give rise to a wide array of phenotypes, including blood cells (Bjornson, 1999). Conversely, bone marrow cells can give rise to blood cells, endothelia, bone, cartilage, fat (Cui, 2000), tendon, lung, liver (Petersen, 1999), muscle, marrow stroma and even brain cells. This indicates that adult stem cells present in numerous tissues may generate multiple cell types, even of different dermal origin. Thus, the classical view that the three germinal layers of ectoderm, endoderm and mesoderm are immutably fixed early during development is not true. As such, the developmental potential of adult somatic stem cells is being reassessed, although the mechanisms that ultimately lead to determining cell fate have yet to be defined. The successful long-term culturing and expansion of such somatic adult stem cells together with their versatility suggests that we can use stem cells, particularly engineered such cells, therapeutically in a spectrum of diseases and disorders of tissues. Preferred engineered stem cells according to the invention are isolated epithelial progenitor cell transfected with a nucleic acid sequence that encodes a functional therapeutic protein or a functional marker protein. Epithelial progenitor cells used according to the invention are characterized, e.g., by their ability to express phenotypic markers of epithelial cells (e.g., cytokeratin and e-cadherin).

The potential application of the technology described herein is in its use for gene therapy, drug delivery and cell replacement therapy in any organ or system of the body. Examples of diseases of the gastrointestinal system that can be treated by the method of the invention include inflammatory bowel disease, gastroparesis, Hirschsprung's disease, infantile hypertrophic pyloric stenosis and diabetes.

Inflammatory bowel disease (IBD) includes a number of diseases that involve the bowel and are characterized by inflammation and ulceration of the small or large bowel. The two most common diseases are ulcerative colitis and Crohn's disease. Approximately 2 million people in the US have IBD and there does appear to be a genetic basis for the disease (Poleski 1996).

Gastroparesis basically means a weak stomach. The condition results in severely impaired emptying of the gastric contents, and can be caused by a number of other diseases such as diabetes. Gastroparesis occurs in approximately 0.3 percent of the US population (Jackson, Gastroenterology 2002).

Hirschsprung's disease occurs in about 1 in every 5000 live births—0.02 percent of the population—and thus is relatively rare (International Foundation for Functional Gastrointestinal Disorders 2002). This disease is caused by the absence of nerve cells in the wall of the bowel, and the result is that stools cannot pass through the bowel, and bowel contents build up behind the obstruction. The only form of current treatment is surgery. (International Foundation for Functional Gastrointestinal Disorders 2002).

Infantile hypertrophic pyloric stenosis (IHPS) affects 1 in 500 infants and is the enlarging of the pylorus, the tube leading from the stomach to the intestine, resulting in a blockage. Symptoms include projectile vomiting, dehydration, and weight loss (Claris Law, Inc. 2002).

Diabetes is characterized by high levels of blood glucose arising from defects in insulin production, action, or both. Type I diabetes develops when there is immunological destruction of the pancreatic β cells and is treated by systemic insulin, usually by daily self-injections. Type I diabetes is thought to account for 5 to 10 percent of all diagnosed cases of diabetes. Type II diabetes accounts for 90 to 95 percent of all diagnosed cases of diabetes, and is treated by diet, exercise, insulin and other drugs (Center for Disease Control and Prevention 2002).

Other examples of diseases and conditions that can be treated according to the method of the invention are given in Table 1, below:

TABLE 1
Gene/Cell Replacement Therapies Possible with the Method of the
Invention

| System | Disease Examples | Examples |
|---|---|---|
| Neurological | Parkinson | Dopaminergic enzymes |
| | Seizure disorders | GABA enzymes |
| | CVA/TBI/SCI | Neuron replacement |
| | | NOS |
| | Psychiatric Disorders | GABA, Dopamine |
| | Somatic Pain | SP |
| | Visceral Pain | CGRP, SP/neurokinins, |
| | | Tryptophan hydroxylase |
| | Depression | Tryptophan hydroxylase |
| | Neurofibromatosis | NF1,2 replacement |
| Cardiovascular | CAD | NOS |
| | | Angiogenic factors |
| | Heart Failure | Myocyte replacement |
| | | Angiogenic Factors |
| | | Positive inotropes |
| | | ACE inhibitors |
| | Hypertension | NOS, ACEI, ANP |
| | Claudication | NOS |
| Genitourinary | ARF | NOS |
| | Impotence | NOS |
| | Hepatorenal Syndrome | NOS |
| | Prostatic hypertrophy | Antiandrogenic factors |
| Pulmonary | Pulmonary HTN | NOS, Prostacyclin |
| | Cystic fibrosis | Airway CFTR |
| | | Replacement |
| | Alpha-1 antitrypsin | Gene therapy |
| | deficiency. | |
| Hepatic | ESLD | Hepatocyte Growth |
| | | Factor |
| | Portal HTN | NOS |
| | Alpha-1 antitrypsin | Gene therapy |
| | deficiency | |
| | Storage: Gaucher's, | Gene therapy |
| | Niemann-Picks | |
| | Hemochromatosis | HFE replacement |
| | Wilson's | ATP7B replacement |
| Gastrointestinal | Achalasia, | NOS |
| | Hirschsprung's | |
| | Pyloric stenosis | NOS |
| | Gastroparesis | NOS, motilin, CCK |
| | | Tryptophan hydroxylase |
| | Bruton's, IgA defic.. | Gene therapy |
| | Absorptive/digestive | Gene therapy |
| | defic. | |
| | IBD | Immunomodulators |
| | | Trefoil proteins |
| | Carbohydrate intolerance | Enzyme delivery |
| | | (lactase) |
| | IBS | Tryptophan hydroxylase |
| Endocrine/ | Type I DM | Insulin, GLP-1 |
| Metabolic | Dwarfism | hGH |
| | Fertility, PCO | Steroid enzyme |
| | G6PD defic. | Enzyme replacement |
| | Abetalipoproteinemia | Enzyme replacement |
| | Glycogen storage | Enzyme replacement |
| | diseases | |
| | FMF | Pyrin |
| | Acatalasemia | Ccatalase replacement |
| | Pheylketonuria | PAH replacement |
| | CF | CFTR replacement |
| | LCAT deficiency | Acyltransferase |
| | | replacement |
| | Pancreatic deficiency | Cell replacement |
| | | Specific zymogens |
| | Mucopolysaccharidoses | Specific enzyme |
| | | replacement |
| Hematologic/ | Anemias | EPO, CSF's |
| Oncologic | Bleeding diatheses | Specific clotting factors |
| | Blood dyscrasias | Immune suppressants |
| | Immune deficiencies | Interferons, lymphokines |
| | Retinoblastomas | RB gene replacement |
| | Hemoglobinopathies | Hgb replacement |
| | Porphyrias | Specific enzyme |
| | | replacement |
| Musculoskeletal | arthritides | Immune suppressants |
| | | Cartilage replacement |
| | | therapy |
| | Duchenne's muscular | Dystrophin |
| | dystrophy | |
| Dermatology | alopecia | Steroid 5-alpha reductase |
| | | Inhibitors |
| | Burn/skin grraft | KGF, EGF |
| Connective Tissue | Marfan's | Fibrillin |
| | achondroplasias | Specific enzymatic |
| | | replacements |
| | Epidermolysis | Specific enzymes |

An ideal application for the method of the invention directed to implanting stem cells for drug-delivery is in the treatment of nitric oxide (NO) deficiency as seen in various human conditions. The model of NO deficiency is ideal to test this widely applicable technology in the digestive system because of the ineffectiveness of traditional pharmacological approaches to treat these conditions, the inventors' experience with producing and maintaining stem cells, the availability of physiologically-relevant animal (genetic knockout) models and our understanding of the relevant physiology of these knockout animals. In humans, a deficiency in NO availability has been implicated in various gastrointestinal motility disorders and in other disorders as indicated in Table 1. However, NO is a readily diffusible and easily inactivated gas, making its local and chronic delivery impossible by conventional pharmacological approaches. As described below, we manipulated stem cells by introducing into them a gene for NO production as a novel means of delivering NO to the gut. The success of this experiment showed that stem cells can indeed be engineered in culture, can be injected locally and can produce NO chronically, e.g., in the adult gastrointestinal tract.

This novel method of gene delivery can produce further clinical benefit: stem cell cycle kinetics, cellular sensitivity to apoptosis and stem cell number, for example, all can be manipulated to improve cancer therapy and prevention. Long-term introduction of genes such as APC (adenomatous polyposis coli) can reduce cancer risk in certain patients. Introducing CFTR (cystic fibrosis transmembrane regulator) can be used to treat cystic fibrosis. Intestinal trefoil factor may be helpful, as a protectant, in treating Inflammatory Bowel Disease. Stem cells, engineered as described, can be implanted readily into a patient in need of therapy, e.g., into the digestive system of the patient endoscopically and/or transcutaneously. The engineered cells can also be administered into other organs or systems, e.g., by other parenteral methods. Optimal dosage and modes of administration can readily be determined by conventional protocols. For example, the stem cell compositions of the invention may be administered in an amount that produces a dosage of the encoded protein of 0.25 μg/kg/day to 5 mg/kg/day, and preferably 1 μg/kg/day to 500 μg/kg/day.

The implanted stem cells of the experiments described herein clearly differentiated into mature cells of the gastrointestinal tract (e.g., epithelial, muscular and neuronal elements), which opens up a wide range of applications to treat inflammatory, degenerative and congenital diseases of the digestive system. For example, any of a number of congenital or acquired defects, e.g., of intestinal transport (absorption and/or secretion), barrier function (inflammatory bowel diseases) motility (neuromuscular degeneration), can potentially be treated by implanting new stem cells into the gastrointestinal/hepatopancreatobiliary organ affected. For example, we have isolated stem cell populations from the intestine, colon, bone marrow and fat using established techniques. We can genetically engineer these stem cells and reintroduce them into the gastrointestinal/hepatopancreatobiliary tracts to treat diseases, for novel drug delivery, cell replacement, and research applications. This is a particularly attractive approach, as, for example, bone marrow can be harvested readily from the same individual, obviating the need for immunosuppression to prevent rejection and graft-versus-host disease. Harvested somatic stem cells can then be engineered to express proteins of interest and re-implanted into organs requiring localized gene therapy, drug delivery or cell replacement.

Our future understanding of gut development and maintenance is enhanced by the availability of stem cells that are easily distinguishable from host cells. To this end, we have engineered stem cells that express reporter genes such as β-galactosidase and a variety of fluorescent proteins, such as green fluorescent protein (GFP). Such tagged cells are invaluable tools, for example, for observing the fate of implanted stem cells, stem cell competition within a crypt, cell regeneration after injury, and observing carcinogenesis. Moreover, tags such as GFP that allow identification of live stem cell progeny in adult tissues permit physiological studies of transfected tissues. Furthermore, the properties of certain fluorescent proteins make them ideal reporters of intracellular physiological events (e.g., GFP is pH sensitive) that can be studied in living tissues by fluorescence imaging.

The following examples are presented to illustrate the advantages of the present invention and to assist one of ordinary skill in making and using the same. These examples are not intended in any way otherwise to limit the scope of the disclosure.

### EXAMPLE I

### Nitric Oxide Delivery to GI Tract via Engineered Stem Cells

The importance of nitric oxide in the regulation of smooth muscle relaxation in the gastrointestinal tract has been well established from pharmacological studies involving various inhibitors of the endogenous enzymes that synthesize this gas. However, such pharmacological agents could not clearly distinguish the three potential enzymatic sources of this mediator, namely neuronal (nNOS, or Type I), endothelial (eNOS, or Type III), and inducible (iNOS, Macrophage or Type II) nitric oxide synthases. The definitive evidence for the key role of nNOS in gastrointestinal motility came from the use of a specific knockout mouse line. Although NO derived from nNOS was deemed to be a key factor in memory formation and neuronal development, the knockout mice lacking nNOS had grossly normal brain anatomy and no clear impediment in learning. The manifest phenotype in these mice was that of an enlarged stomach and muscular hypertrophy that develops particularly in the proximal gastrointestinal tract of adult mice, mimicking the well-described condition of idiopathic hypertrophic pyloric stenosis in humans (Huang, 1993). This enlarged stomach, suggesting gastric motor dysfunction, was specific to mice lacking nNOS, since neither mice lacking eNOS (Huang, 1995) or iNOS (Hickey, 1997) were noted to develop such enlarged stomachs.

We proposed that totipotent embryonic stem cells could be manipulated in vitro to express constitutively an enzyme that produces NO. Although these stem cells are totipotent in embryonic blastocysts, it was unknown whether they would be able survive in and reconstitute the normal adult gut for the long term. However, unlike gene therapy using adenoviral or HSV vectors, in which expression usually wanes over weeks and induces inflammatory reaction, we determined that syngeneic stem cells could be manipulated and injected into the adult gut for chronic stable expression of a particular gene.

Making Tagged Stem Cells. The stem cells used for the following experiments were engineered so that, when injected into a host tissue, they could be easily distinguished from surrounding host tissues. The bacterial β-D-galactosidase gene was chosen as the “tag” because of its ability to turn expressing cells blue easily on histochemical staining and because this stain, formed by an insoluble metal complex, is a lasting marker for the cells. J1 ES cells, which have been used for making a number of knockout mice in the past and which have consistently given high chimerism when injected into blastocysts (an indication of their totipotency), were electroporated with a plasmid containing a fusion gene of the β-galactosidase gene with the neomycin phosphotransferase (resistance) gene (coined “geo”) (Zambrowicz, 1997). Upon transfection by electroporation and after neomycin selection for two weeks, independent colonies of cells were picked and stained for β-galactosidase activity. There was a large variability in the level of staining in these colonies from as low as<2% of the population to as high as 98%).

Alternative “tags” were introduced into stem cells as well. The fluorescent marker GFP was introduced using various methods described in more detail below, including electroporation and chemical transfection. These cells could be maintained in culture with stable expression of GFP fluorescence. These cells were injected into adult digestive organs as described below.

We also successfully derived ES cells from the inner cell mass of embryonic day 2.5 blastocysts of transgenic ROSA26 mice (Jackson Mice, Bar Harbor, Me.) constitutively expressing galactosidase, because the various tissues from these mice were described initially to be nearly 100% positive for X-gal staining (see below). We envisioned that these derived ES cells, called Rosa Stem (RS) cells, would be the ideal tagged injected cells. These “blue cells” are also syngeneic with our host wild type (C57BL/6Jx129SV/J)F1 and with knockout mice deficient in nNOS (Huang, 1993) so that tissue incompatibility and the need for immunosuppression were avoided. These cells were maintained on primary fibroblast cell layers with supplemented leukemia inhibitory factor (Gibco BRL) essentially as described by Robertson (Robertson, 1997) and were confirmed to stain 100% with x-gal, as initially described (Zambrowicz, 1997).

Making stem cells constitutively expressing INOS. A cDNA of the human inducible form of the enzyme (INOS) (Geller, 1993) was cloned into the multiple cloning site of the bicistronic expression vector pIRES-Hyg2 (Clontech, Palo Alto, Calif.). The vector was chosen because this would allow for rapid and efficient selection of positive clones expressing iNOS. The vector includes a single cassette that expresses both INOS and the selection marker (hygromycin resistance gene) from the same strong CMV IE promoter so that virtually all transfected cells expressing the selection marker also express iNOS.

iNOS was chosen because its activity is not dependent upon intracellular calcium/calmodulin, and this enzyme could produce chronically large amounts of NO compared to the constitutive (neuronal and endothelial) isoenzymes. Moreover, the human source for this enzyme may be exploited to distinguish this from the endogenous murine host iNOS in the future. The bicistronic vector was chosen to allow for increased amounts of NO production The iNOS-pIRES vector DNA was transfected by electroporation (Bio-Rad Gene Pulse II) into the ROSA26-derived “blue” stem cells.

After selection with 100, 250 and 400 μg/ml hygromycin B for two weeks, these derived ROSA26-derived stem (RS) cells transfected with the iNOS-pIRES vector demonstrated efficient and stable expression of both β-galactosidase and iNOS on histochemical and immunohistochemical staining. Stable NO production and release into the media were evidenced by an electrochemical NO sensor (ISO-NO MarkII, World Precision Instruments) and by DAF-2DA staining, when compared to untransfected RS cells.

Survival of stem cells in vivo. These iNOS-bearing RS cells (1-5×10⁴ cells) were injected in the adult mouse pylorus using a 32 gauge needle. The tagged “blue” cells persisted in vivo at the site of injection after 1 day (n=3), 7 days (n=5), and 60 days (n=5) in both wild type and nNOS-deficient mice. The injected cells persist for as long a period as we have looked, over a year at this point, with no untoward effects, either on tissue architecture or animal health. In other intestinal segments including ileum and colon, similar results were observed; when injected into the serosal aspect of the ileum and colon, blue cells were seen to persist in the epithelial layer for up to 8 weeks. In all segments, these injected cells continued to express iNOS, as detected by the immunofluorescence marker attributed to this enzyme, and the expressed enzyme produced NO in the injected tissues, as assayed by the NO-trapping DAF-2DA (Calbiochem, La Jolla, Calif.) fluorescent dye. Unlike what has been described by other groups using adenoviral transfection, these cells caused minimal, if any, inflammatory reaction.

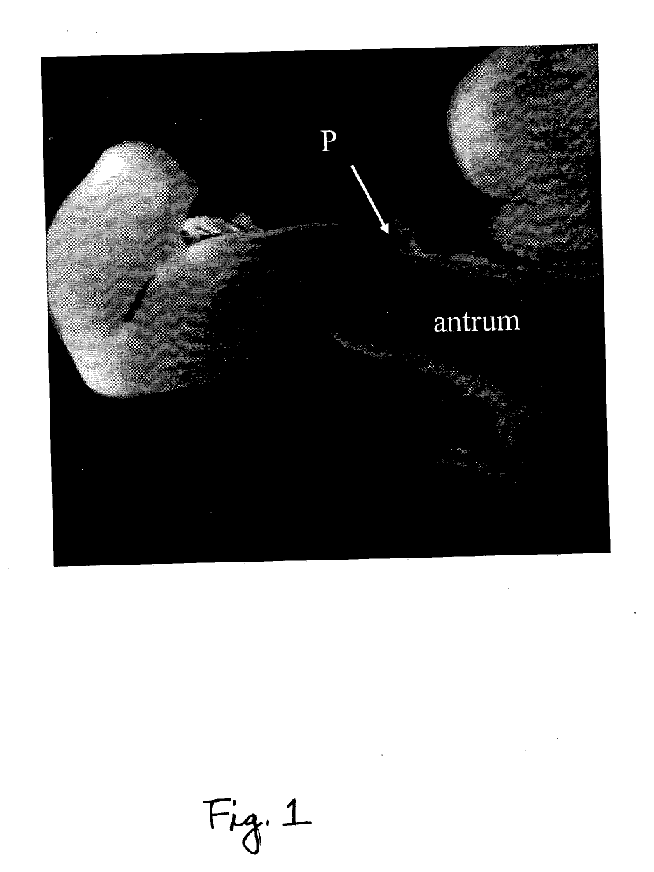

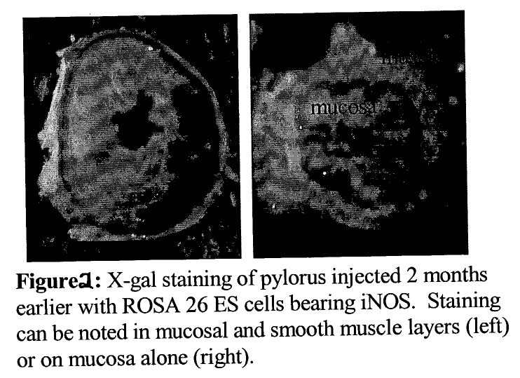

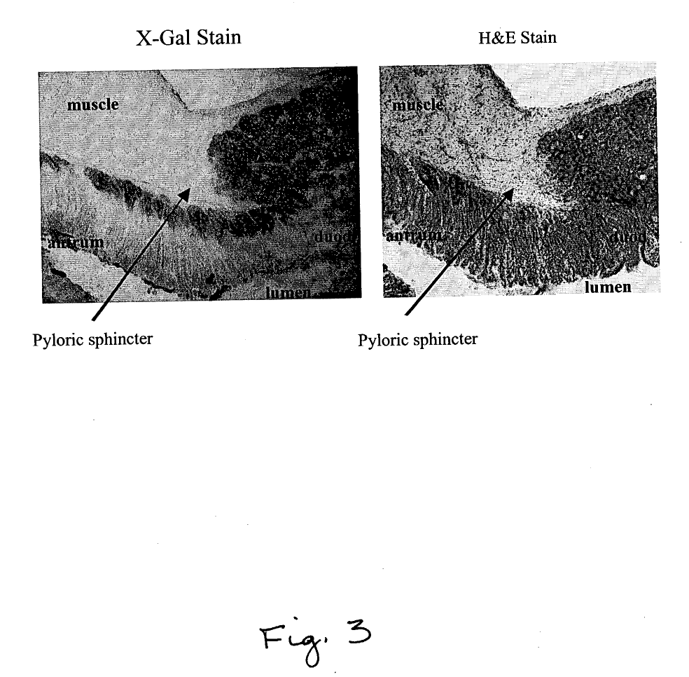

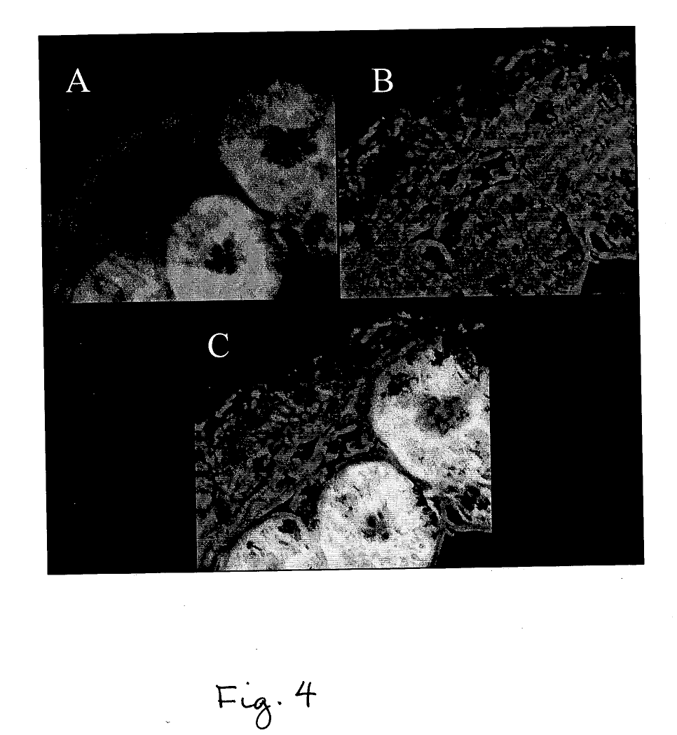

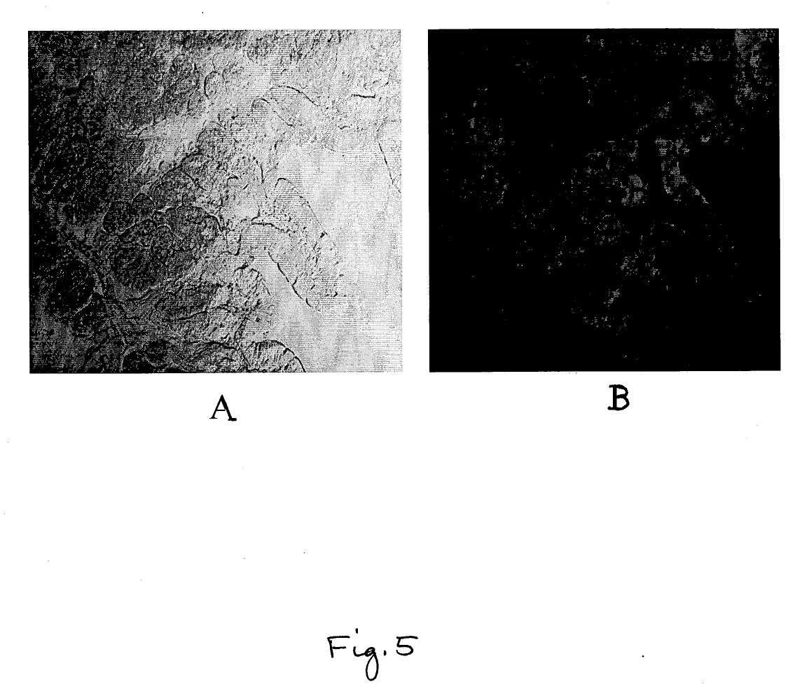

Moreover, these cells can populate mucosa or smooth muscle layers within the pylorus. Injected cells indeed become phenotypically and functionally mature specialized cells of epithelium muscle or neurons in the region near injection. They are clearly observed to express many phenotypic markers of epithelial cells (e.g., cytokeratin and e-cadherin) and to form structures indistinguishable from its milieu (e.g., parietal cells that express gastrin receptors as well as H-K-ATPase). Referring to FIG. 1, it can be seen by X-gal staining of the pylorus that Rosa 26 ES cells bearing INOS are still localized 60 days post-injection. FIGS. 2A and 2B are cross sections of the pylorus in the stained region shown in FIG. 1. Specific staining in the mucosal and smooth muscle layers are shown in FIG. 2A while staining on the mucosa alone is shown in FIG. 2B. FIGS. 3A and 3B show a comparison of X-gal staining (3A) and H&E staining (3B) in the same general region. Staining of the antrum in cross section four weeks after injection with ES cells bearing iNOS is shown in high power detail in FIGS. 4A-4C: (A) antrum stained with epithelial specific anti-e-cadherin antibody; (B) a red pseudocolor DIC image of X-gal staining; (C) antibody and X-gal staining co-localized within gastric glands (yellow). Referring to FIGS. 5A-5B, it can be seen that four weeks after injection of the duodenum (with ES cells bearing iNOS), blue cells are present (5A) largely within glands that are cytokeratin positive (5B).

Similar results to the above were observed with bone marrow and intestine-derived stem cells. These data demonstrate that undifferentiated embryonic and adult somatic stem cells transfected with specific genes survive in vivo in the adult intestine and show great promise for a novel method of drug delivery. This approach appears particularly well suited for such a diffusible, unstable and transiently bioactive compound as nitric oxide.

### EXAMPLE II

### Preparation of Enhanced Fluorescent Stem Cells

We have also created enhanced fluorescent stem cells from the early passage ES cell line J1 used in the laboratory (Mashimo, 1996) by transfecting with the fluorescent marker gene green fluorescent protein (GFP, originally isolated from jellyfish Aequoria victoria) together with the iNOS vector described above. This plasmid produces a protein that brightly fluoresces in response to appropriate wavelength light, and unlike other bioluminescent reporters, does not require additional proteins, substrates, or cofactors to emit light. Thus, it is ideal for monitoring gene expression and protein localization in vivo, in situ, and in real time. Unlike the β-galactosidase reporter, GFP can be detected directly without fixation or disruption, e.g., for studying processing in living cells and whole organisms. These proteins are also stable in living cells, and when fixed, fluorescence can still be detected after several months. Initial electroporation of such an enhanced fluorescent protein in J1 cells and maintenance in culture has confirmed the ease of detection under epifluorescent illumination and ascertained its ability to be expressed in these stem cells.

These improved cells will allow imaging injected cells in live tissues and will be a valuable tool for characterizing the differentiation potential of these cells when injected in specific adult tissue. The conditions for electroporation of a linearized plasmid DNA into the J1 stem cells have been well characterized in the laboratory (see below), and the efficiency of stable incorporation and expression is estimated to be on the order of 10⁻⁵ to 10⁻⁶ colonies per electroporated cell. Our experience with this vector has shown that there is heterogeneity in the intensity of the fluorescence of the cells after electroporation and neomycin selection. Therefore, cells electroporated with the GFP plasmid are allowed to form clonal colonies under neomycin (Geneticin™, Gibco BRL) selection pressure, with the most intensely fluorescent colonies being picked for further expansion. This will greatly select for high expression of the fluorescent marker.

These cells, in turn, will be electroporated with the iNOS-Hygr bicistronic vector described above and grown under hygromycin selection pressure. The final doubly-resistant cells will be confirmed for INOS expression (by immunohistochemistry), NO production (by DAF-2DA and NO electrode sensor, as above), and epifluorescent intensity prior to injection in vivo. Transgenic mice harboring GFP show intense and uniform epiflourescence in virtually every tissue except hair and nails (Ikawa, 1999). Thus, the more stable and enhanced fluorescent proteins are expected to be valuable tags for the stem cells.

### EXAMPLE III

### Non-Embryonic Sources of Stem Cells for Gene Therapy and Drug Delivery

We have harvested non-embryonic stem cells, namely from bone marrow, intestinal mucosa and fat, to assess their chronic survivability and pluripotency in the gastrointestinal tract. We have observed engraftment and differentiation of blue ROSA-26 derived somatic stem cells in intestinal segments as well as in the liver. Future human applicability of stem cell technology for gene therapy or drug delivery will require a histocompatible source that can be derived from adults. Fortunately, there is recent evidence that other tissues, including brain, liver, fat, intestinal mucosa and bone marrow may contain stem cells that have the pluripotency to populate the gastrointestinal tract (Booth, 2000). We can derive these cells from intestinal mucosa and bone marrow of ROSA26 mice for two reasons. First, these tissues have the potential of being procured using less invasively derivable tissues (i.e., by endoscopic mucosal biopsies for intestinal and colonic epithelia, or posterior superior iliac aspirates for bone marrow cells), when these techniques are applied to humans. Second, the stem cells derived from bone marrow and intestinal crypts have been described in the literature as potential sources for repopulating the gut (Krause, 2001).

### DETAILED METHODS

Stem cell culture and electroporation. Embryos, ES cells, and non-embryonic stem cells are cultured in specially formulated ES cell culture medium prepared from HEPES-buffered (20 mM, pH 7.3) Dulbecco's modified Eagles medium (DMEM, high glucose, from Gibco) supplemented with 15% heat inactivated fetal calf serum (HyClone), 0.1 mM non-essential amino acids (100×stock from Gibco), 0.1 mM mercaptoethanol (4 μl per 500 ml medium), antibiotics (penicillin and streptomycin), and Esgro™ (1×10⁶ U/L, Gibco BRL). Normally, 1.5×10⁶ stem cells are seeded in a 25 cm² tissue culture flask that has been gelatinized and plated the day before with mitomycinc-treated or irradiated primary embryonic fibroblasts made in the laboratory. The medium is changed every day. Cells are split 2-3 day after seeding, usually when the flask is about 80% confluent. It is critical to trypsinize cells well to achieve a single cell suspension and to seed cells at appropriate densities to maintain totipotency. To dissociate stem cells completely, the following procedure is used. Cells are washed once with HEPES saline and trypsinized with 0.5 ml Trypsin/EDTA at 37° C. for 4 min. Cells are then detached off the plate and mixed with trypsin/EDTA thoroughly by agitation and incubated for additional 4 min. Five ml medium is added to the cells, and the cells are pipetted several times against the flask to achieve complete dissociation.

Cells are transfected with DNA using electroporation according to standard methods for the gene knockout technology. Briefly, rapidly growing stem cells are trypsinized, counted, washed and resuspended in sterile electroporation buffer containing 20 mM HEPES (pH 7.0), 137 mM NaCl, 5 mM KCl, 0.7 mM Na₂HPO₄, 6 mM glucose, and 0.1 mM β-mercaptoethanol at 2×10⁷ cells/ml. Linearized DNA (using XhoI for the iNOS-Hygr construct, and using NotI for the enhanced fluorescent proteins) is added to the cell suspension at 25 μg/ml. Electroporation is carried out with a BioRad Gene Pulser II using a single pulse at 400 V, 25 mF. Cells are left in the buffer for 10 min at room temperature and then plated at 2-4×10⁶ cells/plate onto 100 mm plates covered with y-irradiated fibroblast cells that are resistant for a selective antibiotic (such as neomycin or hygromycin). The antibiotic-resistant primary embryonic fibroblasts are purchased from Specialty Media (Phillipsburg, N.J.). Thirty-six hours later, medium containing G418 (0.35 mg/ml dry powder, Gibco) or hygromycin (Clontech) is added. Colonies are picked 10-12 days after selection for freezing and expansion.

Stem cells from non-embryonic sources. Bone marrow cells are harvested from ROSA26 mice by flushing the femur and humerus of 3-4 week-old mice with DMEM using a 26 gauge needle and under sterile conditions. Cells are collected, washed once in Hank's buffer, counted, and reconstituted in Hank's buffer at 2.5×10⁶ cells/ml prior to injection. Intestinal and colonic cells are enriched for crypt cells using a method essentially as described by Weiser (Sykes, 1992) As above, crypt-enriched populations of cells are collected, washed in Hank's buffer, counted, and reconstituted in Hank's buffer at 2.5×10⁶ cells/ml prior to injection.

Tissue injection. Overnight-fasted mice are anesthetized with a ketamine/xylazine/acepromazine mixture intramuscularly, and the ventral area is prepped with Povidone-Iodine (10%, Medline Industries, Mundelein, IL) and 70% ethanol. Through a small epigastric incision and with direct vision under a dissection microscope, 5×10⁴ cells in 20 μl are injected in the pyloric region using a 32 gauge needle mounted on a 100 μl Hamilton syringe. This raises a wheal-like bleb spanning the sphincter and involving both the duodenum and the distal antrum. The incision is closed using a single surgical clip, and mice are allowed to recover under a heat lamp. All animal procedures are performed in accordance with institutional review and policies.

Histochemistry and Immunohistochemistry. Mice are euthanized with an overdose of pentobarbital (100 mg/kg, i.p.) and perfused transcardially with ice-cold 10% phosphate-buffered formalin The pyloric sphincter and adjacent 5 mm of tissue on both sides of the sphincter are harvested intact, flushed with normal saline to remove food debris, and fixed in the same fixative overnight at 4° C. After the fixative is removed with three washes of PBS, the lumen is then filled with TBS tissue freezing compound (Triangle Biomedical Sciences, Durham, N.C.) and quickly frozen in TBS using liquid nitrogen. Twelve μm thick sections are made using a Jung cryostat, and slides are stored at −20° C.

Just prior to staining, slides containing the sections are thawed at room temperature. For X-gal histochemical staining, tissues are permeabilized in cold PBS+2 mM MgCl₂+0.01% sodium deoxycholate+0.02% NP40 for ten minutes at 4° C. Slides or whole tissues are then stained in freshly-prepared X-gal solution containing 35 mM K3Fe(CN)₆ potassium ferricyanide, 35 mM K4Fe(CN)₆0.3H₂O potassium ferrocyanide, 2 mM MgCl₂, 0.01% sodium deoxycholate, 0.02% NP40, and 1 mg/ml X-gal (5-bromo-4-chloro-3-indolyl β-D-galactopyranoside) in phosphate buffered saline (PBS). These chemicals are from Sigma (St. Louis, Mo.). The tissues are then rinsed three times in PBS and mounted in gelvatol (Air Products and Chemicals, Inc. Allentown. PA).

The slides containing sections for immunostaining are pre-blocked using host serum of the animal in which secondary antibody was raised (1:10000 dilution v/v) or for direct immunofluorescence using 0.01% (w/v) BSA in PBS. Cytokeratin staining requires trypsin treatment per manufacturer's recommendations. The primary antibodies are used at concentrations suggested by the manufacturers and are allowed to bind overnight at 4° C.

Primary antibodies are incubated overnight at 4° C. The unbound antibodies are removed by three washes in PBS. Sections for indirect staining are then incubated in FITC-conjugated donkey anti-rabbit IgG (1:200, Jackson ImmunoResearch, West Grove, Pa., USA) or Texas Red-conjugated donkey anti-sheep IgG (1:200, Jackson ImmunoResearch) for two hours at room temperature. All the preparations are examined on a Nikon Eclipse T100 inverted microscope and images are captured on a Spot II CCD camera (Diagnostic Instruments, Inc., Sterling Heights, MI).

### REFERENCES

Bianco P, Riminucci M, Gronthos S, Robey P G. Bone marrow stromal stem cells: nature, biology, and potential applications. Stem Cells 19, 180-92. 2001.

Bjornson C R, Rietze R L, Reynolds B A, Magli M C, Vescovi A L. Turning brain into blood: a hematopoietic fate adopted by adult neural stem cells in vivo. Science 283, 534-7. 1999.

Booth C, Potten C S. Gut instincts: thoughts on intestinal epithelial stem cells. J. Clin. Investig. 105, 1493-1499. 2000.

Bortolotti M, Mari C, Lopilato C, Porrazzo G, Miglioli M. Effects of sildenafil on esophageal motility of patients with idiopathic achalasia. Gastroenterology 118, 253-7. 2000.

Bradley A, Robertson E. Embryo-derived stem cells: a tool for elucidating the developmental genetics of the mouse. Curr. Topics in Develop. Biol. 20:357-71, 1986.

Cui Q, Wang G J, Balian G. Pluripotential marrow cells produce adipocytes when transplanted into steroid-treated mice. Connective Tiss. Res. 41, 45-56. 2000.

Dinsmore J, Ratliff J, Deacon T, Pakzaban P, Jacoby D, Galpern W, Isacson O. Embryonic stem cells differentiated in vitro as a novel source of cells for transplantation. Cell Transplantation 5, 131-43. 1996.

Foreman P K, Wainwright M J, Alicke B, Kovesdi I, Wickham T J, Smith J G, Meier-Davis S, Fix J A, Daddona P, Gardner P, Huang M T. Adenovirus-mediated transduction of intestinal cells in vivo. Human Gene Therapy 9, 1313-21. 1998.

Geller D A, Lowenstein C J, Shapiro R A, Nussler A K, Di Silvio M, Wang S C, Nakayama D K, Simmons R L, Snyder S H, Billiar T R. Molecular cloning and expression of inducible nitric oxide synthase from human hepatocytes. Proc. Natl. Acad. Sci. USA 90, 3491-5. 1993.

Hickey M J, Sharkey K A, Sihota E G, Reinhardt P H, Macmicking J D, Nathan C, Kubes P. Inducible nitric oxide synthase-deficient mice have enhanced leukocyte-endothelium interactions in endotoxemia. FASEB J. 11, 955-64. 1997.

Huang P L, Dawson T M, Bredt D S, Snyder S H, Fishman M C. Targeted disruption of the neuronal nitric oxide synthase gene. Cell 75, 1273-86. 1993.

Huang P L, Huang Z, Mashimo H, Bloch K D, Moskowitz M A, Bevan J A, Fishman M C. Hypertension in mice lacking the gene for endothelial nitric oxide synthase. Nature 377, 239-42. 1995.

Ikawa M, Yamada S, Nakanishi T, Okabe M. Green fluorescent protein (GFP) as a vital marker in mammals. Curr. Top. in Dev. Biol. 44:1-20, 1999.

Krause D S, Theise N D, Collector M I, Henegariu O, Hwang S, Gardner R, Neutzel S, Sharkis S J. Multi-organ, multi-lineage engraftment by a single bone marrow-derived stem cell. Cell 105, 369-77. 2001.

Liu S, Qu Y, Stewart T J, Howard M J, Chakrabortty S, Holekamp T F, McDonald J W. Embryonic stem cells differentiate into oligodendrocytes and myelinate in culture and after spinal cord transplantation. Proc. Nat. Acad. Sci. USA 97, 6126-31. 2000.

Lozier J N, Yankaskas J R, Ramsey W J, Chen L, Berschneider H, Morgan R A. Gut epithelial cells as targets for gene therapy of hemophilia. Human Gene Therapy 8, 1481-90. 1997.

Lumelsky N, Blondel O, Laeng P, Velasco I, Ravin R, McKay R. Differentiation of embryonic stem cells to insulin-secreting structures similar to pancreatic islets. Science 292, 1389-94. 2001.

Mashimo H, Kiriyama Y. Embryonic stem cells as a potentially novel drug delivery system of nitric oxide in the murine pylorus. Gastroenterology 120, A87. 2001.

Mashimo H, Wu D C, Podolsky D K, Fishman M C. Impaired defense of intestinal mucosa in mice lacking intestinal trefoil factor. Science 274, 262-5. 1996.

Michalopoulos G K, DeFrances M C. Liver regeneration. Science 276, 60-6. 1997.

Moore M A. Umbilical cord blood: an expandable resource. J. Clin. Investig. 105, 855-6. 2000.

Natarajan D, Grigoriou M, Marcos-Gutierrez CV, Atkins C, Pachnis V. Multipotential progenitors of the mammalian enteric nervous system capable of colonising aganglionic bowel in organ culture. Development 126, 157-68. 1999.

Okabe S, Forsberg-Nilsson K, Spiro A C, Segal M, McKay R D. Development of neuronal precursor cells and functional postmitotic neurons from embryonic stem cells in vitro. Mechanisms of Development 59, 89-102. 1996.

Petersen B E, Bowen W C, Patrene K D, Mars W M, Sullivan A K, Murase N, Boggs S S, Greenberger J S, Goff J P. Bone marrow as a potential source of hepatic oval cells. Science 284, 1168-70. 1999.

Robertson EJ. Derivation and maintenance of embryonic stem cell cultures. Methods Mol. Biol. 75:173-84, 1997.

Sykes D E, Weiser M M. The identification of genes specifically expressed in epithelial cells of the rat intestinal crypts. Differentiation 50, 41-6. 1992.

Vescovi A L, Parati E A, Gritti A, Poulin P, Ferrario M, Wanke E, Frolichsthal-Schoeller P, Cova L, Arcellana-Panlilio M, Colombo A, Galli R. Isolation and cloning of multipotential stem cells from the embryonic human CNS and establishment of transplantable human neural stem cell lines by epigenetic stimulation. Exper. Neurol.156, 71-83. 1999.

Zambrowicz B P, Imamoto A, Fiering S, Herzenberg L A, Kerr W G, Soriano P. Disruption of overlapping transcripts in the ROSA beta geo 26 gene trap strain leads to widespread expression of beta-galactosidase in mouse embryos and hematopoietic cells. Proc. Nat. Acad. Sci. USA 94, 3789-94. 1997.

Zuk P A, Zhu M, Mizuno H, Huang J, Futrell J W, Katz A J, Benhaim P, Lorenz H P, Hedrick M H. Multilineage cells from human adipose tissue: implications for cell-based therapies. Tissue Engineering 7, 211-28. 2001.

While the present invention has been described in conjunction with a preferred embodiment, one of ordinary skill, after reading the foregoing specification, will be able to effect various changes, substitutions of equivalents, and other alterations to the compositions and methods set forth herein. It is therefore intended that the protection granted by Letters Patent hereon be limited only by the definitions contained in the appended claims and equivalents thereof.

What is claimed is:

1. An isolated epithelial progenitor cell transfected with a nucleic acid sequence that encodes a functional protein.

2. An isolated stem cell transfected with a nucleic acid sequence that encodes a nitric oxide synthase.

3. The isolated cell of claim 1, wherein said functional protein is a functional therapeutic protein.

4. The isolated cell of claim 1, wherein said functional protein is a functional marker protein.

5. The isolated cell of claim 1 or claim 2, wherein said cell is of mouse origin.

6. The isolated cell of claim 1 or claim 2, wherein said cell is of human origin.

7. The isolated cell of claim 1 or claim 2, wherein said cell is of embryonic origin.

8. The isolated cell of claim 1 or claim 2, wherein said cell is an autologous somatic cell derived from a tissue of a patient in need of treatment with said cell.

9. The isolated cell of claim 1, wherein said functional protein is nitric oxide synthase.

10. The isolated cell of claim 9, wherein said protein is neuronal nitric oxide synthase.

11. The progenitor cell of claim 9, wherein said enzyme is endothelial nitric oxide synthase.

12. The progenitor cell of claim 9, wherein said enzyme is inducible nitric oxide synthase.

13. The isolated cell of claim 1, wherein said functional protein is green fluorescent protein.

14. A method of introducing a functional protein into a mammal, said method comprising the steps of:
providing a mammal in need of said functional protein; and
administering to said mammal an effective amount of the transfected cell of claim 1 or claim 2.

15. The method of claim 14, wherein said transfected cell is administered into the gastrointestinal tract of said mammal.

16. The method of claim 14, wherein said transfected cell is administered into the liver of said mammal.

17. The method of claim 14, wherein said transfected cell is administered into the pancreas of said mammal.

18. The method of claim 14, wherein said transfected cell is administered into the kidney of said mammal.
